(19) United States
(12) Patent Application Publication
(10) Pub. No.: US 2017/0035358 A1
(43) Pub. Date: Feb. 9, 2017

(54) FORCE SENSING CATHETERS HAVING SUPER-ELASTIC STRUCTURAL STRAIN SENSORS

(71) Applicant: Boston Scientific Scimed Inc., Maple Grove, MN (US)

(72) Inventor: Darrell L. Rankin, Milpitas, CA (US)

(21) Appl. No.: 15/227,231

(22) Filed: Aug. 3, 2016

Related U.S. Application Data
(60) Provisional application No. 62/202,673, filed on Aug. 7, 2015.

(51) Int. Cl. A61B 5/00; A61B 18/14

(52) U.S. Cl. CPC A61B 5/6885; A61B 5/6852; A61B 18/1492; A61B 2018/00351

(57) ABSTRACT

Various embodiments concern a system for measuring a force on a catheter. The catheter can comprise a proximal segment, a distal segment, and an intermediary segment comprising at least one strut. Each strut can extend from the proximal segment to the distal segment. Each strut can be formed from a super-elastic metal alloy material, such as nitinol. The plurality of struts can be configured to resiliently support the distal segment with respect to the proximal segment while permitting relative movement between the distal segment and the proximal segment. The system can comprise control circuitry configured to measure, for each of the plurality of struts, a change in an electrical property of the super-elastic metal alloy material of the strut when the distal segment moves relative to the proximal segment to determine a magnitude and direction of the force.

### CROSS-REFERENCE TO RELATED APPLICATION

This application claims priority to Provisional Application No. 62/202,673, filed Aug. 7, 2015, which is herein incorporated by reference in its entirety.

### TECHNICAL FIELD

The present disclosure relates generally to various force sensing catheter features.

### BACKGROUND

In ablation therapy, it may be useful to assess the contact between the ablation element and the tissue targeted for ablation. In interventional cardiac electrophysiology (EP) procedures, for example, the contact can be used to assess the effectiveness of the ablation therapy being delivered. Other catheter-based therapies and diagnostics can be aided by knowing whether a part of the catheter contacts targeted tissue, and to what degree the part of the catheter presses on the targeted tissue. The tissue exerts a force back on the catheter, which can be measured to assess the contact and the degree to which the catheter presses on the targeted tissue.

The present disclosure concerns, among other things, systems for measuring a force with a catheter.

### SUMMARY

The present disclosure relates to devices, systems, and methods for measuring a force experienced by a catheter.

Example 1 is a system for measuring a force on a catheter, the system including a catheter and control circuitry. The catheter includes a proximal segment, a distal segment, and an intermediary segment. The intermediary segment includes at least one strut. Each strut extends from the proximal segment to the distal segment. Each strut formed from a super-elastic metal alloy material. The at least one strut is configured to resiliently support the distal segment with respect to the proximal segment while permitting relative movement between the distal segment and the proximal segment. The control circuitry is configured to measure, for each of the at least one strut, a change in an electrical property of the super-elastic metal alloy material of the strut when the distal segment moves relative to the proximal segment.

Example 2 is the system of Example 1, wherein the control circuitry is configured to calculate a magnitude and a direction of the force based on the changes in the electrical property of the super-elastic metal alloy material of the at least one strut.

Example 3 is the system of Example 2, further comprising a display, wherein the control circuitry is configured to graphically indicate on the display the magnitude and the direction of the force.

Example 4 is the system of any of Examples 1-3, wherein the change in the electrical property comprises an increase or a decrease in electrical resistance.

Example 5 is the system of any of Examples 1-4, wherein the electrical property is the electrical resistance of the super-elastic metal alloy material.

Example 6 is the system of any of Examples 1-5, wherein the super-elastic metal alloy material is a nickel-titanium alloy.

Example 7 is the system of any of Examples 1-5, wherein the super-elastic metal alloy material is a copper-aluminum-nickel alloy.

Example 8 is the system of any of Examples 1-7, wherein the change in the electrical property of the super elastic metal alloy material is due to the super elastic metal alloy material changing phases during elastic deformation.

Example 9 is the system of Example 8, wherein the changing phases comprising transitioning one or both of into and out of an intermediary phase between austenite and martensite.

Example 10 is the system of any of Examples 1-9, wherein the catheter further comprises a proximal hub located in the proximal segment and a distal hub located in the distal segment, wherein each strut comprises a proximal end that is attached to the proximal hub and a distal end that is attached to the distal hub.

Example 11 is the system of any of Examples 1-10, wherein the at least one strut comprises a plurality of struts.

Example 12 is the system of Example 11, wherein the plurality of struts are configured to mechanically support the distal segment in a base orientation with respect to the proximal segment, flex when the distal segment moves relative to the proximal segment in response to the application of the force and exhibit the change in the electrical property of the super elastic metal alloy material in response to said flexing, and resiliently return the distal segment to the base orientation with respect to the proximal segment once the force has been removed.

Example 13 is the system of any of Examples 11-12, wherein the plurality of struts are arrayed around a longitudinal axis, the longitudinal axis extending through the centers of the proximal segment and the distal segment when the distal segment is in the base orientation with respect to the proximal segment.

Example 14 is a method of measuring an applied force on a catheter within a patient. The catheter includes a proximal segment, a distal segment, and at least one strut that mechanically supports the distal segment with respect to the proximal segment. The method includes measuring an electrical property of each of the at least one strut as the catheter is advanced within the body, detecting a change in the electrical property of each of the at least one strut indicative of the force deflecting the distal segment with respect to the proximal segment, and outputting an indication via a user interface of the force, wherein each of measuring, detecting, and outputting are performed at least in part by control circuitry.

Example 15 is the method of Example 14, wherein each of the at least one strut is formed from nitinol.

Example 16 is a system for measuring a force on a catheter, the system including a catheter and control circuitry. The catheter includes a proximal segment, a distal segment, and an intermediary segment. The intermediary segment includes at least one strut. Each strut extends from the proximal segment to the distal segment. Each strut formed from a super-elastic metal alloy material. The at least one strut is configured to resiliently support the distal segment with respect to the proximal segment while permitting relative movement between the distal segment and the proximal segment. The control circuitry is configured to measure, for each of the at least one strut, a change in an electrical property of the super-elastic metal alloy material of the strut when the distal segment moves relative to the proximal segment.

Example 17 is the system of Example 16, wherein the control circuitry is configured to calculate a magnitude and a direction of the force based on the changes in the electrical property of the super-elastic metal alloy material of the at least one strut.

Example 18 is the system of Example 17, wherein the control circuitry is configured to graphically indicate on the display the magnitude and the direction of the force.

Example 19 is the system of any of Examples 16-18, wherein the electrical property is the electrical resistance of the super-elastic metal alloy material.

Example 20 is the system of any of Examples 16-19, wherein the super-elastic metal alloy material is a nickel-titanium alloy.

Example 21 is the system of any of Examples 16-19, wherein the super-elastic metal alloy material is a copper-aluminum-nickel alloy.

Example 22 is the system of any of Examples 16-21, wherein the change in the electrical property of the super elastic metal alloy material is due to the super elastic metal alloy material changing phases during elastic deformation.

Example 23 is the system of Example 22, wherein the changing phases comprising transitioning one or both of into and out of an intermediary phase between austenite and martensite.

Example 24 is the system of any of Examples 16-23, wherein the at least one strut comprises a plurality of struts.

Example 25 is a system for measuring a force on a catheter, the system including a catheter and control system. The catheter includes a proximal segment, a distal segment, and a spring segment. The spring segment extends from the proximal segment to the distal segment. The spring segment is configured to permit relative movement between the distal segment and the proximal segment in response to application of the force on the distal segment. The spring segment includes at least one structural element. Each structural element extends from the proximal segment to the distal segment. Each structural element is formed from a super-elastic metal alloy material. The at least one structural element is configured to mechanically support the distal segment in a base orientation with respect to the proximal segment, flex when the distal segment moves relative to the proximal segment in response to the application of the force and exhibit a change in an electrical property of the super elastic metal alloy material in response to said flexing, and resiliently return the distal segment to the base orientation with respect to the proximal segment once the force has been removed. The control circuitry is configured to measure, for each of the at least one structural element, the change in the electrical property when the distal segment moves relative to the proximal segment.

Example 26 is the system of Example 25, wherein the change in the electrical property comprises an increase or a decrease in electrical resistance.

Example 27 is the system of any of Examples 25-26, wherein the at least one structural element comprises a plurality of struts.

Example 28 is the system of Example 27, wherein the plurality of struts are arrayed around a longitudinal axis, the longitudinal axis extending through the centers of the proximal segment, the spring segment, and the distal segment when the distal segment is in the base orientation with respect to the proximal segment.

Example 29 is the system of any of Examples 25-28, wherein the catheter further comprises a proximal hub located in the proximal segment and a distal hub located in the distal segment, wherein each strut comprises a proximal end that is attached to the proximal hub and a distal end that is attached to the distal hub.

Example 30 is the system of any of Examples 25-29, wherein the control circuitry is at least partially located within the catheter.

Example 31 is the system of any of Examples 25-30, wherein the control circuitry is configured to calculate, for each of the at least one structural element, an amount of strain that the structural element experiences when the distal segment moves relative to the proximal segment based at least in part on the change in the electrical property.

Example 32 is the system of any of Examples 25-31, wherein the at least one structural element comprises at least three structural elements, and the control circuitry is configured to calculate a magnitude and a direction of the force based on the changes in the electrical property for the at least three structural elements

Example 33 is the system of Example 32, further comprising a display, wherein the control circuitry is configured to graphically indicate on the display the magnitude and the direction of the force.

Example 34 is a method of measuring an applied force on a catheter within a patient. The catheter includes a proximal segment, a distal segment, and at least one strut that mechanically supports the distal segment with respect to the proximal segment. The method includes measuring an electrical property of each of the at least one strut as the catheter is advanced within the body, detecting a change in the electrical property of each of the at least one strut indicative of the force deflecting the distal segment with respect to the proximal segment, and outputting an indication via a user interface of the force, wherein each of measuring, detecting, and outputting are performed at least in part by control circuitry.

Example 35 is the method of Example 34, wherein each of the at least one strut is formed from nitinol.

While multiple embodiments are disclosed, still other embodiments of the present invention will become apparent to those skilled in the art from the following detailed description, which shows and describes various illustrative embodiments of the present disclosure. Accordingly, the drawings and detailed description are to be regarded as illustrative in nature and not restrictive.

### BRIEF DESCRIPTION OF THE DRAWINGS

FIGS. 1A-C show a system for measuring a force with a catheter in accordance with various embodiments of this disclosure.

FIG. 2A shows a circuit diagram for measuring a change in an electrical property of a structural element.

FIG. 2B shows a block diagram of circuitry for controlling various functions described herein.

FIG. 3 shows a detailed perspective view of a distal end of a catheter in accordance with various embodiments of this disclosure.

FIG. 4 shows a perspective view of the inside of a catheter in accordance with various embodiments of this disclosure.

FIG. 5 shows a side view of the inside of a catheter in accordance with various embodiments of this disclosure.

FIG. 6 shows a cross sectional view taken along line AA of FIG. 5.

FIG. 7 shows a perspective view of hubs in accordance with various embodiments of this disclosure.

FIG. 8A-C shows a side view of a strut in different states of strain in accordance with various embodiments of this disclosure.

While the scope of the present disclosure is amenable to various modifications and alternative forms, specific embodiments have been shown by way of example in the drawings and are described in detail below. The intention, however, is not to limit the scope of the invention to particular embodiments described and/or shown. On the contrary, the invention is intended to cover all modifications, equivalents, and alternatives falling within the scope of the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivated tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radio frequency ablation, cyroablation, microwave ablation, laser ablation, and surgical ablation, among others. While cardiac ablation therapy is referenced herein as an exemplar, various embodiments of the present disclosure can be directed to ablation of other types of tissue and/or to non-ablation diagnostic and/or catheters that deliver other therapies.

Ideally, an ablation therapy can be delivered in a minimally invasive manner, such as with a catheter introduced to the heart through a vessel, rather than surgically opening the heart for direct access (e.g., as in a maze surgical procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a single catheter and keep track of all of the information that the catheter collects or is otherwise associated with the procedure. In particular, it can be challenging to determine the location of the therapy element (e.g., the proximity to tissue), the quality of a lesion, and whether the tissue is fully lesioned, under-lesioned (e.g., still capable of generating and/or conducting unwanted electrical signals), or over-lesioned (e.g., burning through or otherwise weakening the cardiac wall). The quality of the lesion can depend on the degree of contact between the ablation element and the targeted tissue. For example, an ablation element that is barely contacting tissue may not be adequately positioned to deliver effective ablation therapy. Conversely, an ablation element that is pressed too hard into tissue may deliver too much ablation energy or cause a perforation.

The present disclosure concerns, among other things, methods, devices, and systems for assessing a degree of contact between a part of a catheter (e.g., an ablation element) and tissue. Knowing the degree of contact, such as the magnitude and the direction of a force generated by contact between the catheter and the tissue, can be useful in determining the degree of lesioning of the targeted tissue. Information regarding the degree of lesioning of cardiac tissue can be used to determine whether the tissue should be further lesioned or whether the tissue was successfully ablated, among other things. Additionally or alternatively, an indicator of contact can be useful when navigating the catheter because a user may not feel a force being exerted on the catheter from tissue as the catheter is advanced within a patient, thereby causing vascular or cardiac tissue damage or perforation.

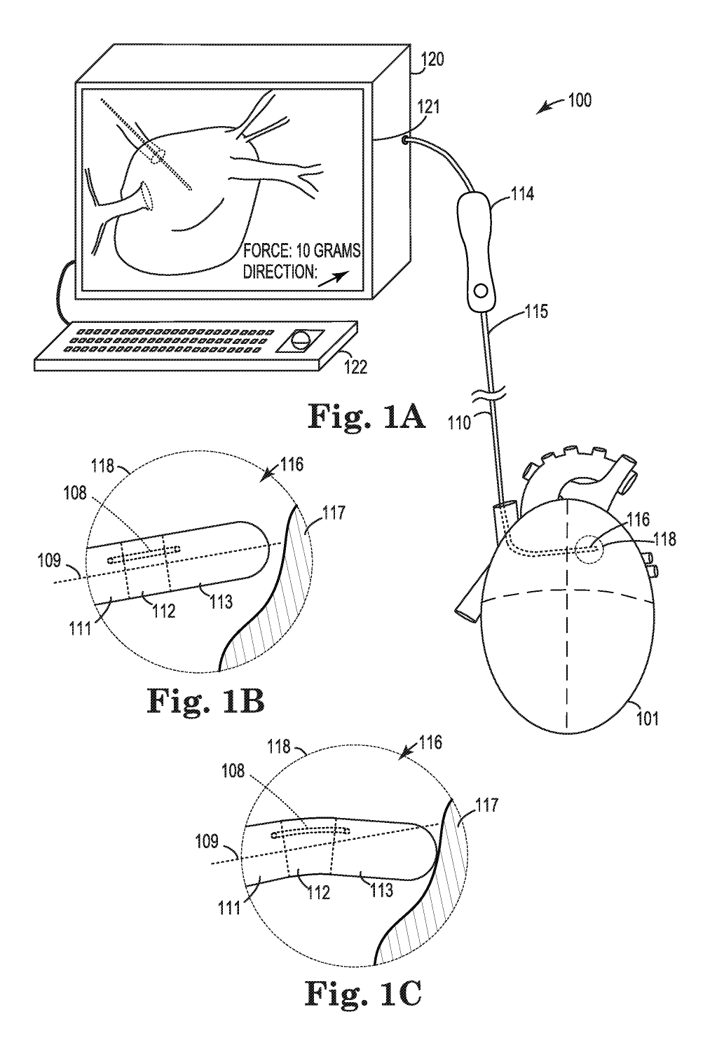

FIGS. 1A-1C illustrate an embodiment of a system 100 for sensing data from inside the body and/or delivering therapy. For example, the system 100 can be configured to map cardiac tissue and/or ablate the cardiac tissue, among other options. The system 100 includes a catheter 110 connected to a control unit 120 via handle 114. The catheter 110 can comprise an elongated tubular member having a proximal end 115 connected with the handle 114 and a distal end 116 configured to be introduced within a heart 101 or other area of the body. As shown in FIG. 1A, the distal end 116 of the catheter 110 is within the left atrium of heart 101.

As shown in FIGS. 1B and 1C, the distal end 116 of the catheter 110 includes a proximal segment 111, a spring segment 112, and a distal segment 113. The distal segment 113, or any other segment, can be in the form of an electrode configured for sensing electrical activity, such as electrical cardiac signals. Such an electrode (or other electrode on the catheter 110) can additionally or alternatively be used to deliver ablative energy to tissue.

The proximal segment 111, the spring segment 112, and the distal segment 113 can be coaxially aligned with each other in a base orientation as shown in FIG. 1B. Specifically, each of the proximal segment 111, the spring segment 112, and the distal segment 113 are coaxially aligned with a common longitudinal axis 109. The longitudinal axis 109 can extend through the radial center of each of the proximal segment 111, the spring segment 112, and the distal segment 113, and can extend through the radial center of the distal end 116 as a whole. In some embodiments, the coaxial alignment of the proximal segment 111 with the distal segment 113 can correspond to the base orientation. As shown, the distal end 116, at least along the proximal segment 111, the spring segment 112, and the distal segment 113, extends straight. In some embodiments, this straight arrangement of the proximal segment 111, the spring segment 112, and the distal segment 113 can correspond to the base orientation.

The proximal segment 111, the spring segment 112, and the distal segment 113 can be mechanically biased to assume the base orientation. Specifically, a structural element 108 can reside within the distal end 116 of the catheter 110. The structural element 108 can extend from the proximal segment 111, through the spring segment 112, to the distal segment 113. While a single structural element 108 is shown in FIGS. 1B-C, a plurality of structural elements can be provided along the same longitudinal location as the structural element 108, and can be configured in any manner as the structural element 108. The structural element 108 can mechanically support the distal segment 113 relative to the proximal segment 111. For example, the structural element 108 can provide most or all of the mechanical support that holds the distal segment 113 in the base orientation with respect to the proximal segment 111. It is the structural element 108 which can provide the spring properties of the spring segment 112. A proximal end of the structural element 108 can be anchored in the proximal segment 111 while a distal end of the structural element 108 can be anchored within the distal segment 113. For example the proximal end of the structural element 108 can be rigidly attached to material within the proximal segment 111 while the distal end of the structural element 108 can be rigidly attached to material within the distal segment 113. The structural element 108 can be in the form of a wire, a helically wound coil, a ribbon, or other shape. As shown, the structural element 108 can be generally elongated from the proximal segment 111 to the distal segment 113.

The structural element 108 can be formed from a super-elastic metal alloy, such as a nickel-titanium alloy (e.g., nitinol), a copper-zinc-aluminum alloy, a copper-aluminum alloy, or a copper-aluminum-nickel alloy. Super-elastic metal alloys can be useful in catheters because of such metals exhibit large elastic deformation ranges and therefore are resilient. Such resiliency can return the shape of the distal end 116 of the catheter 110 to its nominal base orientation after deflection.

The catheter 110 includes force sensing capabilities. For example, the catheter 110 is configured to sense a force due to engagement with tissue 117. The distal segment 113 can be relatively rigid while segments proximal of the distal segment 113 can be relatively flexible. In particular, the spring segment 112 may be more flexible than the distal segment 113 and the proximal segment 111 such that when the distal end 116 of the catheter 110 engages tissue 117, the spring segment 112, as shown in FIG. 1C, bends. For example, the distal end 116 of the catheter 110 can be generally straight as shown in FIG. 1B. When the distal segment 113 engages tissue 117, the distal end 116 of the catheter 110 can bend at the spring segment 112 such that the distal segment 113 moves relative to the proximal segment 111. As shown in FIGS. 1B and 1C, the normal force from the tissue moves the distal segment 113 out of coaxial alignment (e.g., with respect to the longitudinal axis 109) with the proximal segment 111 while the spring segment 112 bends. As such, proximal segment 111 and the distal segment 113 may be stiff to not bend due to the force while the spring segment 112 may be less stiff and bend to accommodate the force exerted on the distal end 116 of the catheter 110.

The structural element 108 can be used to determine the magnitude and the direction of the force due to engagement with the tissue 117. Super-elastic metal alloys can be induced to transition between martensite and austenite phases based on a change in temperature, thus providing shape memory effects. Super-elastic metal alloys have slip planes such that the material changes phases under elastic deformation. Super-elastic metal alloys can be forced to transition between martensite and austenite phases by induction of stress in the material. For example, a super-elastic metal alloy material may be in the austenite phase when unstressed but will transform to the martensite phase above a critical stress (e.g., during deformation). The material can transition back to the austenite phase once the stress is released. Between the martensite and austenite phases is an unstable transition area phase which is referred to as the “R” phase herein. One remarkable aspect of the R phase is an electrical property of the super-elastic metal alloy material changes as it transitions through the R phase. Specifically, the resistivity of the super-elastic metal alloy material increases as it transitions through the R phase under increasing stress. Various embodiments of the present disclosure capitalize on this phenomenon by measuring an electrical property of a structural element formed by a super-elastic metal alloy to determine the strain that the structural element is undergoing. As such, the structural element can serve multiple purposes including mechanically supporting parts of the distal end 116 of the catheter 110 while also functioning as a strain sensor.

As shown in FIG. 1C, the distal segment 113 has moved relative to the proximal segment 111, thereby straining the structural element 108. Specifically, the structural element 108 is shown to be bending relative to the state of the structural element 108 in FIG. 1B. Such bending can change an electrical property of the structural element 108, as discussed above. For example, straining may change the electrical resistivity of the structural element 108. Conductors, such as copper wires, can be attached to the proximal and distal ends of the structural element 108 to run current through the structural element 108. The signal passed to the structural element 108 can be measured by circuitry determine whether the resistance of the structural element 108 change over time, indicative of the structural element 108 having been strained. Therefore, a measured increase in electrical resistivity of the structural element 108 can indicate that the distal segment 113 moved relative to the proximal segment 111. The magnitude of the force moving the distal segment 113 can be calculated using Hooke's law, wherein the strain of the structural element 108 is proportional to the forced placed on element.

The control unit 120 of the system 100 includes a display 121 (e.g., LCD) for displaying information. The control unit 120 further includes a user input 122 which can comprise one or more buttons, toggles, a track ball, a mouse, touchpad, or the like for receiving user input. The user input 122 can additionally or alternatively be located on the handle 114. The control unit 120 can contain control circuitry for performing the functions referenced herein. Some or all of the control circuitry can alternatively be located within the handle 114.

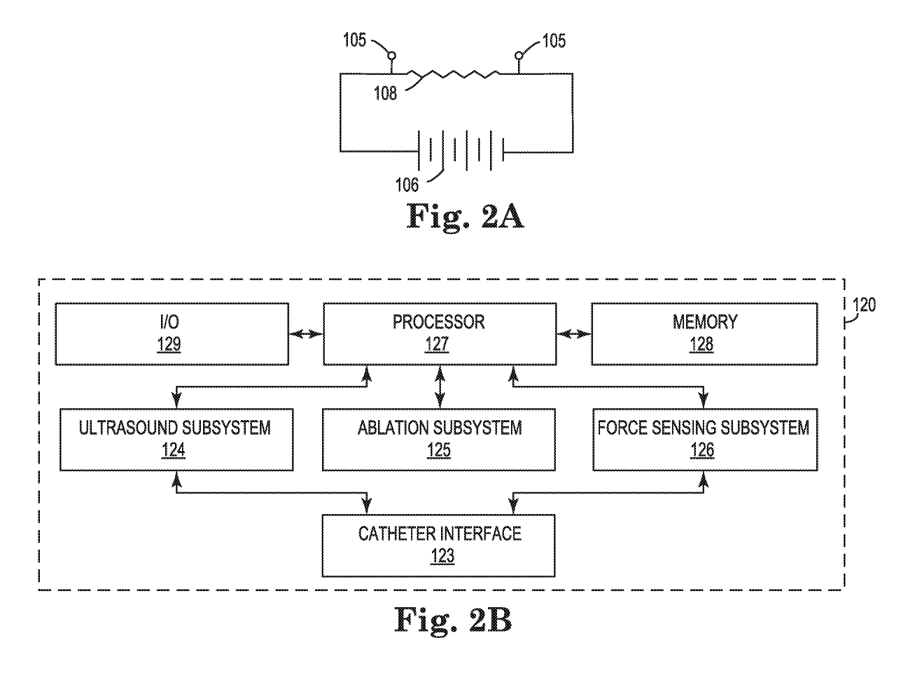

FIG. 2A shows a circuit diagram for measuring electrical property of the structural element 108. Structural element 108 is represented as a resistor because, as discussed previously, the change in electrical property can be the resistance of the structural element 108. The power source 106 can provide constant voltage or current across the structural element 108. The change in resistance of the structural element 108 can be measured from the nodes 105 by a change in voltage or current based on the changing resistance of the structural element 108.

FIG. 2B illustrates a block diagram showing an example of control circuitry which can perform functions referenced herein. This or other control circuitry can be housed within control unit 120, which can comprise a single housing or multiple housings among which components are distributed. Control circuitry can additionally or alternatively be housed within the handle 114. The components of the control unit 120 can be powered by a power supply (not shown), known in the art, which can supply electrical power to any of the components of the control unit 120 and the system 100. The power supply can plug into an electrical outlet and/or provide power from a battery, among other options.

The control unit 120 can include a catheter interface 123. The catheter interface 123 can include a plug which receives a cord from the handle 114. The catheter 110 can include multiple conductors (not illustrated but known in the art) to convey electrical signals between the distal end 116 and the proximal end 115 and further through the handle 114 to the catheter interface 123. It is through the catheter interface 123 that the control unit 120 (and/or the handle 114 if control circuitry is included in the handle 114) can send electrical signals to any element within the catheter 110 and/or receive an electrical signal from any element within the catheter 110. The catheter interface 123 can conduct signals to or from any of the components of the control unit 120.

The control unit 120 can include an ultrasound subsystem 124 which includes components for operating the ultrasound functions of the system 100. While the illustrated example of control circuitry shown in FIG. 2B includes the ultrasound subsystem 124, it will be understood that not all embodiments may include ultrasound subsystem 124 or any circuitry for imaging tissue. The ultrasound subsystem 124 can include a signal generator configured to generate a signal for ultrasound transmission and signal processing components (e.g., a high pass filter) configured to filter and process reflected ultrasound signals as received by an ultrasound transducer in a sense mode and conducted to the ultrasound subsystem 124 through a conductor in the catheter 110. The ultrasound subsystem 124 can send signals to elements within the catheter 110 via the catheter interface 123 and/or receive signals from elements within the catheter 110 via the catheter interface 123.

The control unit 120 can include an ablation subsystem 125. The ablation subsystem 125 can include components for operating the ablation functions of the system 100. While the illustrated example of control circuitry shown in FIG. 2B includes the ablation subsystem, it will be understood that not all embodiment may include ablation subsystem 125 or any circuitry for generating an ablation therapy. The ablation subsystem 125 can include an ablation generator to provide different therapeutic outputs depending on the particular configuration (e.g., a high frequency alternating current signal in the case of radiofrequency ablation to be output through one or more electrodes). Providing ablation energy to target sites is further described, for example, in U.S. Pat. No. 5,383,874 and U.S. Pat. No. 7,720,420, each of which is expressly incorporated herein by reference in its entirety for all purposes. The ablation subsystem 125 may support any other type of ablation therapy, such as microwave ablation. The ablation subsystem 125 can deliver signals or other type of ablation energy through the catheter interface 123 to the catheter 110.

The control unit 120 can include a force sensing subsystem 126. The force sensing subsystem 126 can include components for measuring a force experienced by the catheter 110. The force sensing subsystem 126 can include some of the components shown in FIG. 2A. Such components can include signal processors, analog-to-digital converters, operational amplifiers, transistors, comparators, and/or any other circuitry for conditioning and measuring one or more signals. The force sensing subsystem 126 can send signals to elements within the catheter 110 via the catheter interface 123 and/or receive signals from elements within the catheter 110 via the catheter interface 123.

Each of the ultrasound subsystem 124, the ablation subsystem 125, and the force sensing subsystem 126 can send signals to, and receive signals from, the processor 127. The processor 127 can be any type of processor for executing computer functions. For example, the processor 127 can execute program instructions stored within the memory 128 to carry out any function referenced herein, such as determine the magnitude and direction of a force experienced by the catheter 110.

The control unit 120 further includes an input/output subsystem 129 which can support user input and output functionality. For example, the input/output subsystem 129 may support the display 121 to display any information referenced herein, such as a graphic representation of tissue, the catheter 110, and a magnitude and direction of the force experienced by the catheter 110, among other options. Input/output subsystem 129 can log key and/or other input entries via the user input 122 and route the entries to other circuitry.

A single processor 127, or multiple processors, can perform the functions of one or more subsystems, and as such the subsystems may share control circuitry. Although different subsystems are presented herein, circuitry may be divided between a greater or lesser numbers of subsystems, which may be housed separately or together. In various embodiments, circuitry is not distributed between subsystems, but rather is provided as a unified computing system. Whether distributed or unified, the components can be electrically connected to coordinate and share resources to carry out functions.

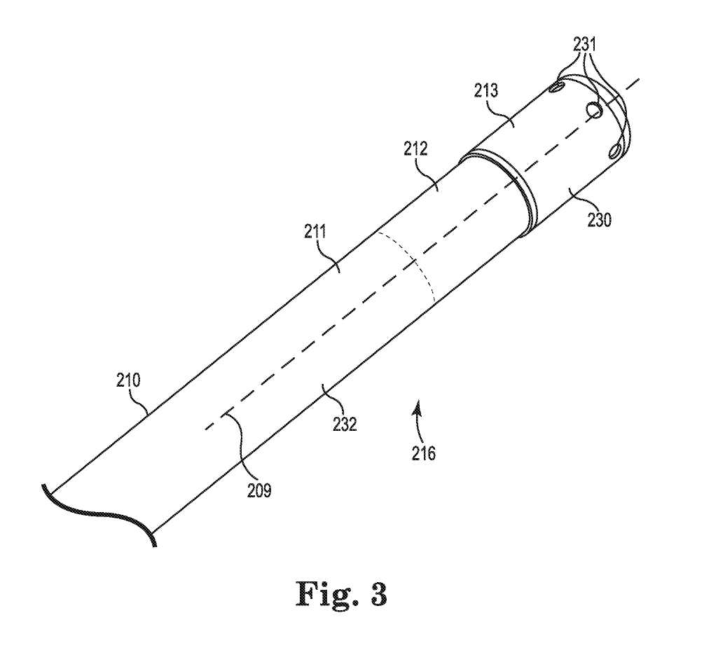

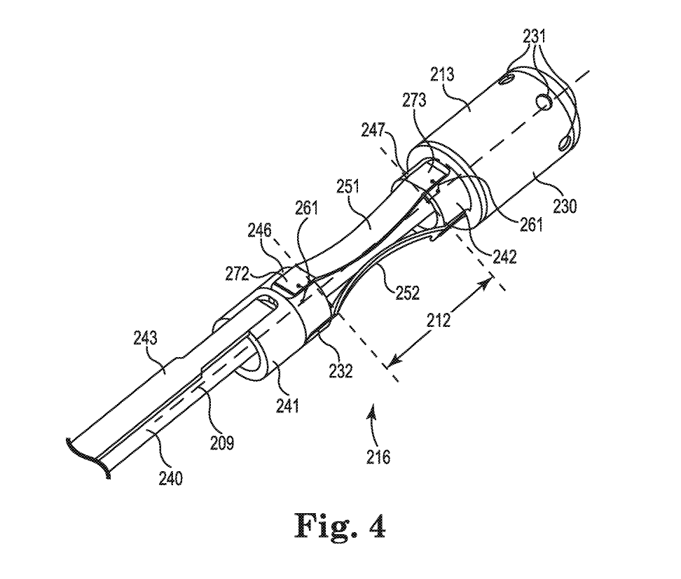

FIG. 3 illustrates a detailed view of a distal end 216 of a catheter 210. The catheter 210 can be used in a system similar to the system 100 shown in FIGS. 1A-2B. It is noted that elements having similar two digit base reference numbers (e.g., 1XY and 2XY) can be similar to the counterpart embodiments shown and described herein unless shown or described to be incompatible. The embodiment shown in FIGS. 3-8C can be similar, unless otherwise noted, to the embodiment of FIGS. 1A-2B and can share components and functions that may be discussed in connection with one embodiment but not shown or discussed (for the sake of brevity) with the other. FIG. 3 shows a catheter shaft 232. The catheter shaft 232 can extend from the distal segment 213 to a handle (e.g., handle 114), and thus can define an exterior surface of the catheter 210 along the spring segment 212, the proximal segment 211, and further proximally to the proximal end 215. The catheter shaft 232 can be a polymeric tube formed from various polymers, such as polyurethane, polyamide, polyether block amide, silicone, and/or other materials. In some embodiments, the catheter shaft 232 may be relatively flexible, and at least along the spring segment 212 may not provide any material mechanical support to the distal segment 213 (e.g., facilitated by thinning of the wall of the catheter shaft 232 along the spring segment 212).

As shown, the proximal segment 211 can be proximal and adjacent to the spring segment 212. The length of the proximal segment 211 can vary between different embodiments, and can be five millimeters to five centimeters, although different lengths are also possible. The length of the spring segment 212 can also vary between different embodiments, and can be dependent on the length of underlying struts as will be further discussed herein. The spring segment 212 is adjacent to the distal segment 213. As shown in FIG. 3, the distal segment 213 can be defined by an electrode 230. The electrode 230 can be an ablation electrode. In some other embodiments, the distal segment 213 may not be electrode. The electrode 230 can be in a shell form which can contain other components. The electrode 230 can include a plurality of ports 231. One or more ultrasonic transducers, housed within the electrode 230, can transmit and receive signals through the ports 231 or through additional dedicated holes in the tip shell. Additionally, or in place of the transducers, one or more miniature electrodes may be incorporated into the tip shell assembly

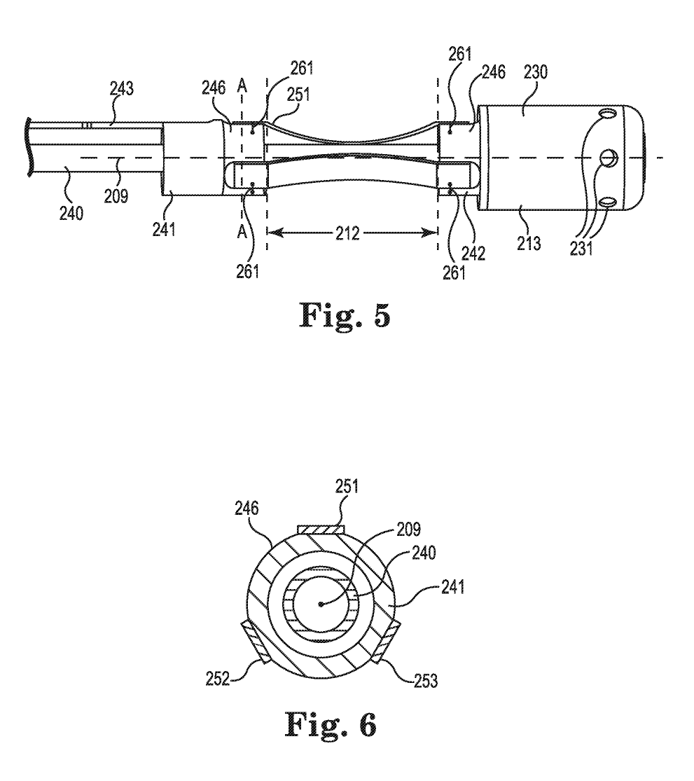

FIG. 4 shows the catheter 210 after the removal of the catheter shaft 232 to expose various components that underlie the catheter shaft 232. FIG. 5 shows a side view of the distal end 216 of the catheter 210 with the shaft 232 removed, as with FIG. 4. The removal of the catheter shaft 232 exposes structural and force sensing components. The components can include a proximal hub 241, a distal hub 242, and a plurality of struts 251-253 (strut 253 shown in FIG. 6) that bridge between the proximal hub 241 and the distal hub 242. The proximal hub 241 and the distal hub 242 can be respective rings to which the plurality of struts 251-253 is attached. One or both of the proximal hub 241 and the distal hub 242 can be formed from electrically insulative material, such as polymer (e.g., polyethylene or polyether etherketone), and/or a composite or ceramic material.

The proximal hub 241 and the distal hub 242 can be coaxially aligned with respect to the longitudinal axis 209. For example, the longitudinal axis 209 can extend through the respective radial centers of each of the proximal hub 241 and the distal hub 242. One or more inner tubes 240 (one shown) can extend through the catheter 210 (e.g., to the handle 114), through the proximal hub 241 and the distal hub 242. The inner tube 240 can include one or more lumens within which one or more conductors (e.g., conductors 261) can extend from the proximal end 215 to the distal segment 213, such as for connecting with one or more electrical elements (e.g., ultrasound transducer, electrode, struts 251-253, or other component). Coolant fluid can additionally or alternatively be routed through the inner tube 240, or through an additional inner tube 240. In various embodiments, the catheter 210 is open irrigated (e.g., through the plurality of ports 231) to allow the coolant fluid to flow out of the distal segment 213. Various other embodiments concern a non-irrigated catheter 210.

A tether 243 can attach to a proximal end of the proximal hub 241. The tether 243 can attach to a deflection mechanism within a handle to cause deflection of the distal end 216. A knob, slider, or plunger on a handle may be used to create tension or slack in the tether 243.

As shown in FIGS. 4 and 5, the spring segment 212 can extend from a distal edge of the proximal hub 241 to a proximal edge of the distal hub 242. As such, the proximal hub 241 can be part of, and may even define the length of, the proximal segment 211. Likewise, the distal hub 242 can be part of the distal segment 213. The proximal hub 241 and the distal hub 242 can be stiffer than the plurality of struts 251-253 such that a force directed on the distal segment 213 causes the distal end 216 to bend along the plurality of struts 251-253 (the spring segment 212 specifically) rather than along the distal segment 213 or the proximal segment 211. The spring segment 212 can receive most or all of its mechanical support from the plurality of struts 251-253. For example, the distal segment 213 may be mechanically maintained in a base orientation with respect to the longitudinal axis 209 mostly or entirely by the plurality of struts 251-253 (e.g., wherein all other components contribute negligible or no mechanical support of the distal segment 213 relative the proximal segment 211).

The proximal hub 241 includes an attachment portion 246. The attachment portion 246 can be on a distal side of the proximal hub 241. Proximal portions of the plurality of struts 251-253 can be attached to the attachment portion 246. For example, a proximal portion 272 of the strut 251 can be attached to the attachment portion 246 of the proximal hub 241. The distal hub 242 can include an attachment portion 247. The attachment portion 247 can be on a proximal side of the distal hub 242. Distal ends of the plurality of struts 251-253 can be attached to the attachment portion 247. For example, a distal portion 273 of the strut 251 can be attached to the attachment portion 247 of the distal hub 242. The length of the spring segment 212 may be defined as the length of the plurality of struts 251-253 that is not overlapped by either of the proximal hub 241 or the distal hub 242 because this is the portion of the distal end 216 which is configured to bend due to a force.

Each of the plurality of struts 251-253 can be similar to the structural element 108 in form and/or function. Each strut 251-253 can be a respective unitary piece of metal formed from a super-elastic metal alloy material, such as a nickel-titanium alloy (e.g., nitinol), a copper-zinc-aluminum alloy, a copper-aluminum alloy, or a copper-aluminum-nickel alloy. The plurality of struts 251-253 can therefore be formed of a super-elastic metal alloy material and can exhibit the mechanical and electrical character characteristics discussed herein. For example, the plurality of struts 251-253 can mechanically support the distal segment 213 relative to proximal segment 211 while also functioning as individual strain sensors by changing in an electrical property under strain. Conductors 261 can be attached to opposite proximal and distal ends of the struts 251-253, respectively, to run current through the struts 251-253 to measure the change in the electrical property. For example, a conductor 261 can connect to the proximal portion 272 of the strut 251 while another conductor 261 can connect to the distal portion 273 of the strut 251. The conductors can be routed through holes in the proximal hub 241 and the distal hub 242 and into the inner tube 240 then extend within a lumen of the inner tube 240 to a proximal end of the catheter 210 for delivering signals to and/or from control circuitry. The conductors 261 can be copper wires insulated by a polymer coating.

The plurality of struts 251-253 are circumferentially arrayed around the longitudinal axis 209 such that one or more of the struts will be compressed when the distal segment 213 moves relative to the proximal segment 211 while one or more of the other struts will be stretched when the distal segment 213 moves relative to the proximal segment 211. Which struts elongate or compress depends on the direction of the force. If the force had a different direction, a different one or more of the struts will be compressed while a different one or more of the struts will be stretched. Based on the different amounts of stretching and compressing of the struts 251-253, and which struts 251-253 compress and which struts 251-253 elongate, the magnitude and direction of force can be determined by the force sensing subsystem 126. In particular, each of the plurality of struts 251-253 can undergo a phase change to exhibit a measurable change in electrical resistivity indicative of bending of the strut. Each strut 251-253 can sense the strain (compression or stretching) in the struts itself to determine the magnitude and direction of the force.

FIG. 6 shows a cross-sectional view along line AA of FIG. 5. In particular, the cross-sectional view cuts through the proximal hub 241. All three struts 251-253 are shown in FIG. 6. As shown, the struts 251-253 are circumferentially arrayed around the proximal hub 241 (and likewise can be circumferentially arrayed around the distal hub 242 in the same manner), the inner tube 240, and the longitudinal axis 209. The respective centers of the three struts 251-253 can be separated by 120 degrees, for example. It will be understood that a different number of struts can alternatively be provided, such as two, four, five, or more. The struts can be evenly spaced circumferentially around the proximal hub 241 (and likewise around the distal hub 242 in the same manner), the inner tube 240, and/or the longitudinal axis 209.

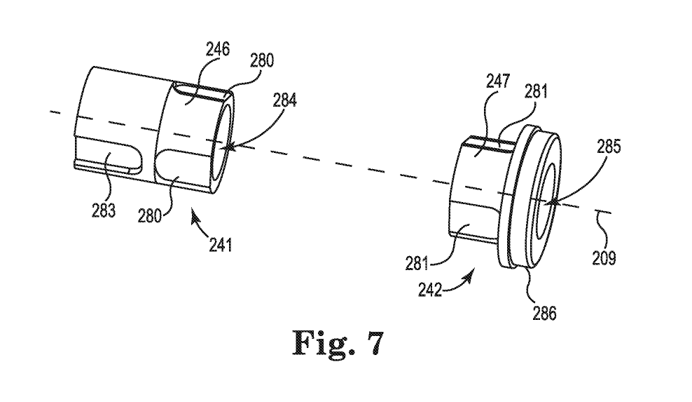

FIG. 7 shows perspective views of the proximal hub 241 and the distal hub 242 in respective isolation. As such, the proximal hub 241 includes a lumen 284 and the distal hub 242 includes a lumen 285. Conductors, the inner tube 240 or other elements can extend through the lumens 284, 285. The proximal hub 241 includes a plurality of attachment surfaces 280. As shown, each attachment surface 280 can be flat while the rest of the attachment portion 246 is relatively round. As such, the attachment portion 246 can comprise alternating flat and round sections that extend around the circumference of the proximal hub 241. Each attachment surface 280 can serve as a surface to interface with a flat, proximal portion of a respective one of the struts 251-253. The struts 251-253 can be attached to the attachment portion 246 at such attachment surfaces 280. The struts 251-253 can be attached to the proximal hub 241 by an adhesive (e.g., epoxy), welding, and/or riveting. In some embodiments, a collar may be placed over the proximal ends of the struts 251-253 to pinch the proximal ends of the struts 251-253 between the collar and the proximal hub 241 to attach the struts 251-253 to the proximal hub 241.

The distal hub 242 includes a plurality of attachment surfaces 281. Each attachment surface 281 can be flat while the rest of the attachment portion 247 can be relatively round. As such, the attachment portion 247 can comprise alternating flat and round sections that extend around the circumference of the distal hub 242. Each attachment surface 281 can serve as a surface to interface with a flat, distal portion of a respective one of the struts 251-253. The struts 251-253 can be attached to the attachment portion 247 at such attachment surfaces 281. The struts 251-253 can be attached to the distal hub 242 by an adhesive (e.g., epoxy), welding, and/or riveting. In some embodiments, a collar may be placed over the distal ends of the struts 251-253 to pinch the distal ends of the struts 251-253 between the collar and the distal hub 242 to attach the struts 251-253 to the distal hub 242. The proximal hub 241 and the distal hub 242 in the form from electrically insulative material to electrically isolate the plurality of struts 251-253 from each other to maintain signaling integrity for each strut.

The struts 251-253 can be circumferentially arrayed around each of the proximal hub 241 and the distal hub 242. The circumference (or diameter) of the attachment portion 246 of the proximal hub 241 can be equal to the circumference (or diameter) of the attachment portion 247 of the distal hub 242. The attachment of the struts 251-253 to the proximal hub 241 and the distal hub 242 can secure the distal hub 242 to the proximal of 241 while allowing movement of the distal hub 242 relative to the proximal hub 241. Furthermore, the struts 251-253 can be structurally resilient to return the distal hub 242 back to the base orientation (e.g., coaxial with longitudinal axis 209) with respect to the proximal hub 241 once an external force to the catheter has been removed.

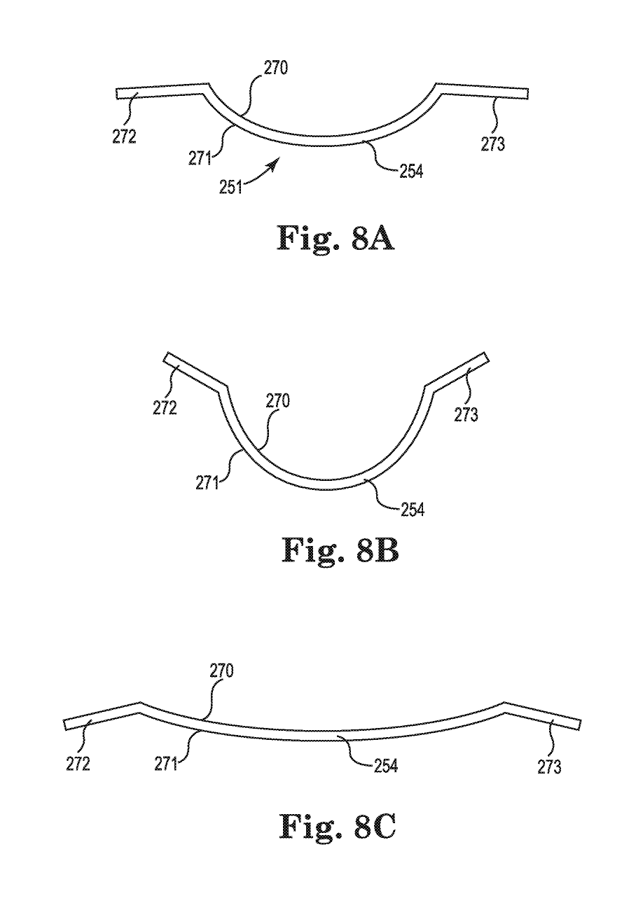

FIGS. 8A-C show isolated views of different states of the strut 251. While strut 251 is shown, FIGS. 8A-C and associated discussion can represent the mechanics of any strut referenced herein. Being that the struts 251-253 can be identical, the views of strut 251, and the discussion herein, can apply to any of the struts. As shown, the strut has a proximal portion 272, a distal portion 273, and a bend 254 which extends from the proximal portion 272 to the distal portion 273. As shown, the strut 251 has the profile of a rectangular strip. The strut 251 includes the first side 271 and a second side 270 opposite the first side 271. The first side can extend over each of the proximal portion 272, the bend 270, and the distal portion 273. Likewise the second side 270 can extend over each of the proximal portion 272, the bend 270, and the distal portion 273. While these struts 251 include the bend 254, various struts may not include a bend and maybe flat.

The proximal portion 272 can be flat, the distal portion 273 can be flat, and the bend 254 can be in a nonplanar configuration. The bend 254 of the strut 251 can extend proximally to the proximal portion 272 and distally to the distal portion 273. For example, the proximal portion 272 can be coplanar with the distal portion 273, while the bend 254 can be curved therebetween.

Considering FIGS. 7 and 8A-8C together, the proximal portion 272 and the distal portion 273 can be shaped to interface with the attachment surfaces 280, 281 of the proximal hub 241 and the distal hub 242, respectively, for attachment therebetween. The proximal portion 272 can contact, and be directly attached to, the attachment portion 246 (e.g., a flat portion of the attachment portion 246). For example, the proximal portion 272 can be adhered with an adhesive, can be welded, or can be riveted, among other options, to the proximal hub 241 (e.g., to attachment surface 281 of the attachment portion 246). The distal portion 273 can contact, and be directly attached to, the attachment portion 247 (e.g., a flat portion of the attachment portion 247). For example, the distal portion 273 can be adhered with an adhesive, can be welded, or can be riveted, among other options, to the distal hub 242 (e.g., to attachment surface 182 of the attachment portion 247).

It is noted that the first side 271 is radially inward facing while the second side 270 is radially outward facing in FIGS. 4 and 5. In this way, the struts 251-253 bow radially inward. The bowing of the bend 254 radially inward means that the strut 251 will further bow inward when compressed, thereby keeping the profile of the assembly compact. The inner tube 240 or other element may serve to bottom out the bowing of the struts 251-253 (e.g., by contact between the bends of the struts and the inner tube 240 or other element) to prevent potentially damaging over-compression. The struts 251-253 may alternatively bow radially outward, however bending of the struts 251-253 outward increases the overall radius of the array of struts 251-253 thereby increasing the hoop strength of the array of struts 251-253. Being that it may not be desirable for the array of struts 251-253 to increase in strength when attempting to measure a force, it may be preferable to have the pre-formed bends to bow radially inward rather than outward.

FIG. 8A shows the strut 251 in an unstrained state. For example, the strut 251 can be pre-biased to assume the shape shown in FIG. 8A. FIG. 8B shows the strut 251 in a stretched state. FIG. 8C shows the strut 251 in a compressed state. If the strut 251 is placed in either of the stretched state or compressed state by the force placed on the catheter 210, the strut 251 will resiliently return to the pre-biased state shown in FIG. 8A once the force is removed. As such, the plurality of struts 251-253 can structurally support the distal segment 213 from the proximal segment 211, can allow the distal segment 213 to move relative to the proximal segment 211 based on a force exerted on the distal segment 213, and can resiliently return the distal segment 213 to its original base orientation with respect to the proximal segment 211 once the force has been removed. It is noted that the plurality of struts 251-253 may provide most or all of the mechanical support that holds the distal segment 213 in the base orientation with respect to the proximal segment 211 and resiliently return the distal segment 213 to the base orientation with respect to the proximal segment 211 after removal of the force. The compression and elongation of the struts 251-253 during such relative movement of the distal segment 213 and the proximal segment 211 can be measured to determine the magnitude of the force and the direction force, as discussed herein. A constant signal can be fed to each of the struts 251-253 via conductors 261 to establish a baseline resistance or other electrical parameter value. Deviation from this baseline indicates compression or elongation of the strut. For example, elongation may be represented by an increase in electrical resistance relative to the baseline, and the amount of increase in the resistance can be proportional to the amount of elongation to allow calculation of the amount of elongation of the strut. Likewise compression may be represented by a decrease in electrical resistance relative to the baseline, and the amount of decrease in resistance can be proportional to the amount of the compression to allow calculation of the amount of compression of the strut.

If the force exerted on the distal segment 213 is coaxial with the longitudinal axis 209, then each of the struts 251-253 will compress in equal amounts. The struts 251-253 will exhibit equal amounts of dimensional change in the bends of the struts 251-253. Based on these equal changes, the control circuitry can determine a magnitude and direction of the force. The magnitude of the force can be calculated using Hooke's law, wherein the displacement of a spring element (e.g., strut 251) is proportional to the force placed on the element, based on a predetermined constant. Being that the displacements are equal for each of the struts 251-253, the control circuitry can determine that the force is coaxial with the longitudinal axis 209. If the force is not coaxial with the longitudinal axis 209, then one or more of the struts will be in compression (e.g., by as shown in FIG. 8B) while one or more of the struts are in tension (e.g., as shown in FIG. 8C) relative to the state shown in FIG. 8A. The distal segment 213 will tend to curl or shift radially away from the force with respect to the proximal segment 211. Therefore, the one or more struts in tension indicate the direction from which the force is coming while the one or more struts in compression indicate the opposite direction (in which the force is being applied). Based on this, the direction (e.g., unit vector) of the force can be determined by the control circuitry.

The pre-bending of the strut 251 ensures that the bend 254 will experience much if not all of the overall bending of the strut 251. This results in improved predictable and consistent bending profile, ideal for measuring. The bend 254 may be the only portion of the strut 251 that bends, therefore the change in resistivity of the material of the strut 251 may be limited to the bend 254. As noted previously, the respective bends of the struts 251-253 can be coextensive with the spring segment 212 such that most or all of the bending in the distal end 216 is captured by the bends and measured by the change in electrical property discussed herein.

Once assembled, the catheter 210 may undergo a calibration step, either at a factory or just before use by a physician. In such a step, a plurality of forces of known magnitude and direction can be placed, in sequence, on the distal segment 213 to move the distal segment 213 relative to the proximal segment 211 while the struts 251-253 output signals or otherwise exhibit changes in on electrical property indicative of the bending of the struts 251-253. A table can be generated indicating a separate entry for each force. Thereafter, a force of unknown magnitude and/or direction can be analyzed by comparing signals output from the struts 251-253 to the values of the table to identify the best match. An algorithm can identify which entry from the calibration data has three (or other number depending on the number of struts) change-in-resistance values best matching the current change-in-resistance values. The magnitude and direction of the known force from the calibration step can be indicated as the magnitude and direction currently being experienced. In some cases, a mathematical relationship can be generated based on the linearity of Hooke's law, wherein a limited number of calibration steps are performed to determine the change-in-resistance, or other parameter, and interpolation and/or extrapolation can be computed based on these calibration values. For example, the spring constant can be determined for a strut such that a subsequent elongation or contraction amount can be multiplied by the spring constant to determine the magnitude of the force acting on the distal segment 213 (and thus the strut). The deflection of multiple struts can be factored for determining an overall magnitude and direction for the force.

The magnitude can be represented in grams or another measure of force. The magnitude can be presented as a running line graph that moves over time to show new and recent force values. The direction can be represented as a unit vector in a three dimensional reference frame (e.g., relative to an X, Y, and Z axes coordinate system). In some embodiments, a three dimensional mapping function can be used to track the three dimensional position of the distal end 216 of the catheter 210 in the three dimensional reference frame. Magnetic fields can be created outside of the patient and sensed by a sensor that is sensitive to magnetic fields within the distal end 216 of the catheter 210 to determine the three dimensional position of the distal end 216 of the catheter 210 in the three dimensional reference frame. The direction can be represented relative to the distal end 216 of the catheter 210. For example, a line projecting to, or from, the distal segment 213 can represent the direction of the force relative to the distal segment 213. Such representations can be made on a display as discussed herein.

In some embodiments, the magnitude and direction of the force that are indicated to the user indicates the magnitude and the direction of a force that acts on the distal segment 213. This force typically results from the distal segment 213 pushing against tissue. Therefore, the force acting on the distal segment 213 may be a normal force resulting from the force that the distal segment 213 exerts on the tissue. In some embodiments, it is the force acting on the distal segment 213 that is calculated and represented to a user. Additionally or alternatively, it is the force that the distal segment 203 applies to tissue that is calculated and represented to the user.

The magnitude and direction of the force can be used for navigation by providing an indicator when the catheter encounters tissue and/or for assessing the lesioning of tissue by determining the degree of contact between the lesioning element and the tissue, among other options. In some embodiments, a force under 10 grams is suboptimal for lesioning tissue (e.g., by being too small) while a force over 40 grams is likewise suboptimal for lesioning tissue (e.g., by being too large). Therefore, a window between 10 and 40 grams may be ideal for lesioning tissue and the output of the force during lesioning may provide feedback to the user to allow the user to stay within this window. Of course, other force ranges ideal for lesioning may be used.

The techniques described in this disclosure, including those attributed to a system, control unit, control circuitry, processor, or various constituent components, may be implemented wholly or at least in part, in hardware, software, firmware or any combination thereof. A processor, as used herein, refers to any number and/or combination of a microprocessor, a digital signal processor (DSP), an application specific integrated circuit (ASIC), a field-programmable gate array (FPGA), microcontroller, discrete logic circuitry, processing chip, gate arrays, and/or any other equivalent integrated or discrete logic circuitry. As part of control circuitry, at least one of the foregoing logic circuitry can be used, alone or in combination with other circuitry, such as memory or other physical medium for storing instructions, to carry about specified functions (e.g., a processor and memory having stored program instructions executable by the processor for determining a magnitude and a direction of a force exerted on a catheter). The functions referenced herein may be embodied as firmware, hardware, software or any combination thereof as part of control circuitry specifically configured (e.g., with programming) to carry out those functions, such as in means for performing the functions referenced herein. The steps described herein may be performed by a single processing component or multiple processing components, the latter of which may be distributed among different coordinating devices. In this way, control circuitry may be distributed between multiple devices. In addition, any of the described units, modules, subsystems, or components may be implemented together or separately as discrete but interoperable logic devices of control circuitry. Depiction of different features as modules, subsystems, or units is intended to highlight different functional aspects and does not necessarily imply that such modules or units must be realized as hardware or software components and/or by a single device. Rather, specified functionality associated with one or more module, subsystem, or units, as part of control circuitry, may be performed by separate hardware or software components, or integrated within common or separate hardware or software components of control circuitry.

When implemented in software, the functionality ascribed to the systems, devices, and control circuitry described in this disclosure may be embodied as instructions on a physically embodied computer-readable medium such as RAM, ROM, NVRAM, EEPROM, FLASH memory, magnetic data storage media, optical data storage media, or the like, the medium being physically embodied in that it is not a carrier wave, as part of control circuitry. The instructions may be executed to support one or more aspects of the functionality described in this disclosure.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof.

What is claimed is:

1. A system for measuring a force on a catheter, the system comprising:
a catheter comprising:
a proximal segment;
a distal segment; and
an intermediary segment comprising at least one strut, each strut extending from the proximal segment to the distal segment, each strut formed from a super-elastic metal alloy material, the at least one strut configured to resiliently support the distal segment with respect to the proximal segment while permitting relative movement between the distal segment and the proximal segment; and
control circuitry configured to measure, for each of the at least one strut, a change in an electrical property of the super-elastic metal alloy material of the strut when the distal segment moves relative to the proximal segment.

2. The system of claim 1, wherein the control circuitry is configured to calculate a magnitude and a direction of the force based on the changes in the electrical property of the super-elastic metal alloy material of the at least one strut.

3. The system of claim 2, further comprising a display, wherein the control circuitry is configured to graphically indicate on the display the magnitude and the direction of the force.

4. The system of claim 1, wherein the electrical property is the electrical resistance of the super-elastic metal alloy material.

5. The system of claim 1, wherein the super-elastic metal alloy material is a nickel-titanium alloy.

6. The system of claim 1, wherein the super-elastic metal alloy material is a copper-aluminum-nickel alloy.

7. The system of claim 1, wherein the change in the electrical property of the super elastic metal alloy material is due to the super elastic metal alloy material changing phases during elastic deformation.

8. The system of claim 7, wherein the changing phases comprising transitioning one or both of into and out of an intermediary phase between austenite and martensite.

9. The system of claim 1, wherein the at least one strut comprises a plurality of struts.

10. A system for measuring a force on a catheter, the system comprising:
a catheter comprising:
a proximal segment;
a distal segment; and
a spring segment that extends from the proximal segment to the distal segment, the spring segment configured to permit relative movement between the distal segment and the proximal segment in response to application of the force on the distal segment, the spring segment comprising at least one structural element, each structural element extending from the proximal segment to the distal segment, each structural element formed from a super-elastic metal alloy material, the at least one structural element configured to:
mechanically support the distal segment in a base orientation with respect to the proximal segment,
flex when the distal segment moves relative to the proximal segment in response to the application of the force and exhibit a change in an electrical property of the super elastic metal alloy material in response to said flexing, and
resiliently return the distal segment to the base orientation with respect to the proximal segment once the force has been removed; and
control circuitry configured to measure, for each of the at least one structural element, the change in the electrical property when the distal segment moves relative to the proximal segment.

11. The system of claim 10, wherein the change in the electrical property comprises an increase or a decrease in electrical resistance.

12. The system of claim 10, wherein the at least one structural element comprises a plurality of struts.

13. The system of claim 12, wherein the plurality of struts are arrayed around a longitudinal axis, the longitudinal axis extending through the centers of the proximal segment, the spring segment, and the distal segment when the distal segment is in the base orientation with respect to the proximal segment.

14. The system of claim 10, wherein the catheter further comprises a proximal hub located in the proximal segment and a distal hub located in the distal segment, wherein each strut comprises a proximal end that is attached to the proximal hub and a distal end that is attached to the distal hub.

15. The system of claim 10, wherein the control circuitry is at least partially located within the catheter.

16. The system of claim 10, wherein the control circuitry is configured to calculate, for each of the at least one structural element, an amount of strain that the structural element experiences when the distal segment moves relative to the proximal segment based at least in part on the change in the electrical property.

17. The system of claim 10, wherein the at least one structural element comprises at least three structural elements, and the control circuitry is configured to calculate a magnitude and a direction of the force based on the changes in the electrical property for the at least three structural elements

18. The system of claim 17, further comprising a display, wherein the control circuitry is configured to graphically indicate on the display the magnitude and the direction of the force.

19. A method of measuring an applied force on a catheter within a patient, the catheter comprising a proximal segment, a distal segment, and at least one strut that mechanically supports the distal segment with respect to the proximal segment, the method comprising:
measuring an electrical property of each of the at least one strut as the catheter is advanced within the body;
detecting a change in the electrical property of each of the at least one strut indicative of the force deflecting the distal segment with respect to the proximal segment; and
outputting an indication via a user interface of the force, wherein each of measuring, detecting, and outputting are performed at least in part by control circuitry.

20. The method of claim 19, wherein each of the at least one strut is formed from nitinol.
